(19) United States
(12) Patent Application Publication
(10) Pub. No.: US 2009/0259206 A1
(43) Pub. Date: Oct. 15, 2009

(54) Catheterization Device for a Urine Bag

(76) Inventors: Yu-Chun KAI, Changhua City (TW); Hsiu-Feng TENG, Fongyuan City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/100,478

(22) Filed: Apr. 10, 2008

(51) Int. Cl. A61F 5/458

(52) U.S. Cl. 604/352

(57) ABSTRACT

An catheterization device for a urine bag comprises a urine bag coupled with a sleeve unit, wherein the urine bag includes a conduit connected on the top end thereof and a tubular member connected on the lower end thereof and having a control valve mounted thereon, and the sleeve unit includes a sleeve body, a stop piece, and a double sided tape. The sleeve body includes an opening arranged on one end thereof and a connecting post extending from another end thereof for connecting with the conduit of the urine bag, and includes a ventilation hose extending from one side of the outer periphery thereof. The stop piece includes a through hole arranged at the center thereof and a slot disposed on one side thereof for communicating with the through hole, and the double sided tape is provided to adhere a male genital and the sleeve body.

### BACKGROUND OF THE INVENTION

1. Field of the Invention

The present invention relates to a catheterization device, and more particularly to a catheterization device for a urine bag that may be fitted onto the male genital comfortably.

2. Description of the Prior Arts

A conventional urine bag is used to help the patient who is sick with urine incontinence. As shown in FIG. 1, a conventional urine bag is attached onto the patient's leg and comprises a storage bag 1 including a tubular member 2 with a control valve connected on the lower end thereof and a conduit 3 coupled on the upper end thereof. The conduit 3 includes a soft flexible sleeve member 4 coupled on another end thereof. In operation, the sleeve member 4 is fitted onto the male genital so that the urines flow into the storage bag 1 through the sleeve member 4 and the conduit 3. Thereafter, after the storage bag 1 is full of the urines, they will be discharged through the tubular member 2.

However, such a conventional urine bag still has the following disadvantages:

1. The user feels uncomfortable and the sleeve member is easy to remove from the male genital, and a vacuum generates to stop the urines flowing into the storage bag 1 while in use.

2. The air permeability is poor because the sleeve member 4 completely covers the genital completely, thereby causing bacteria affection.

The present invention has arisen to mitigate and/or obviate the afore-described disadvantages.

### SUMMARY OF THE INVENTION

The primary object of the present invention is to provide a catheterization device for a urine bag that may be fitted onto the male genital securely and comfortably.

Further object of the present invention is to provide a catheterization device for a urine bag that may prevent from a vacuum, discharging urines to the urine bag effectively.

Another object of the present invention is to provide a catheterization device for a urine bag that may prevent from bacteria affection.

In accordance with one aspect of the present invention, there is provided a catheterization device for a urine bag comprising

a urine bag coupled with a sleeve unit and received in a storage bag for being attached onto a user's leg, wherein the urine bag includes a conduit connected on the top end thereof and having a connector coupled onto one end thereof, and includes a tubular member connected on the lower end thereof and having a control valve mounted thereon for discharging the urine stored in the urine bag, and the sleeve unit includes a sleeve body, a stop piece, and a double sided tape. The sleeve body is made of a soft flexible material and formed in a cylinder shape, and includes an opening arranged on one end thereof and a connecting post extending from another end thereof, and includes a ventilation hose extending from one side of the outer periphery thereof. The stop piece is formed in the shape of a lamination and includes a through hole arranged at the center thereof and a slot disposed on one side thereof for communicating with the through hole, and the double sided tape is made of anti-allergic material, such as emulsion, the storage bag includes a bore formed on the upper end thereof for receiving the urine bag, and includes two bands, each adhered on one side thereof.

The present invention will become more obvious from the following description when taken in connection with the accompanying drawings, which show, for purpose of illustrations only, the preferred embodiment in accordance with the present invention.

### BRIEF DESCRIPTION OF THE DRAWINGS

FIG. 1 is a perspective view of a conventional urine bag;

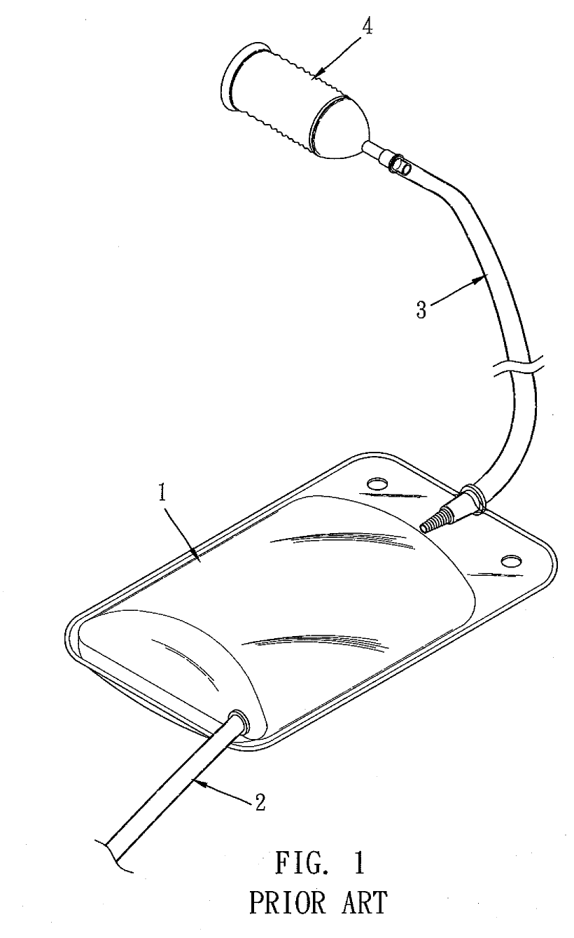

FIG. 2 is a perspective view illustrating the exploded components of a catheterization device for a urine bag according to the present invention;

FIG. 3 is a perspective view illustrating the assembly of the catheterization device for the urine bag according to the present invention;

FIG. 4 is a cross sectional view illustrating the assembly of the catheterization device for the urine bag according to the present invention;

FIG. 5 is a perspective view illustrating the operation of the catheterization device for the urine bag according to the present invention;

FIG. 6 is another perspective view illustrating the operation of the catheterization device for the urine bag according to the present invention;

FIG. 7 is a side plan view illustrating the operation of the catheterization device for the urine bag according to the present invention;

FIG. 8 is another side plan view illustrating the operation of the catheterization device for the urine bag according to the present invention;

FIG. 9 is also another perspective view illustrating the operation of the catheterization device for the urine bag according to the present invention;

FIG. 10 is a perspective view illustrating a ventilation hose of the present invention being closed by using a tie member;

FIG. 11 is a perspective view illustrating the ventilation of the present invention being closed by using a torn segment of a double sided tape.

### DETAILED DESCRIPTION OF THE PREFERRED EMBODIMENT

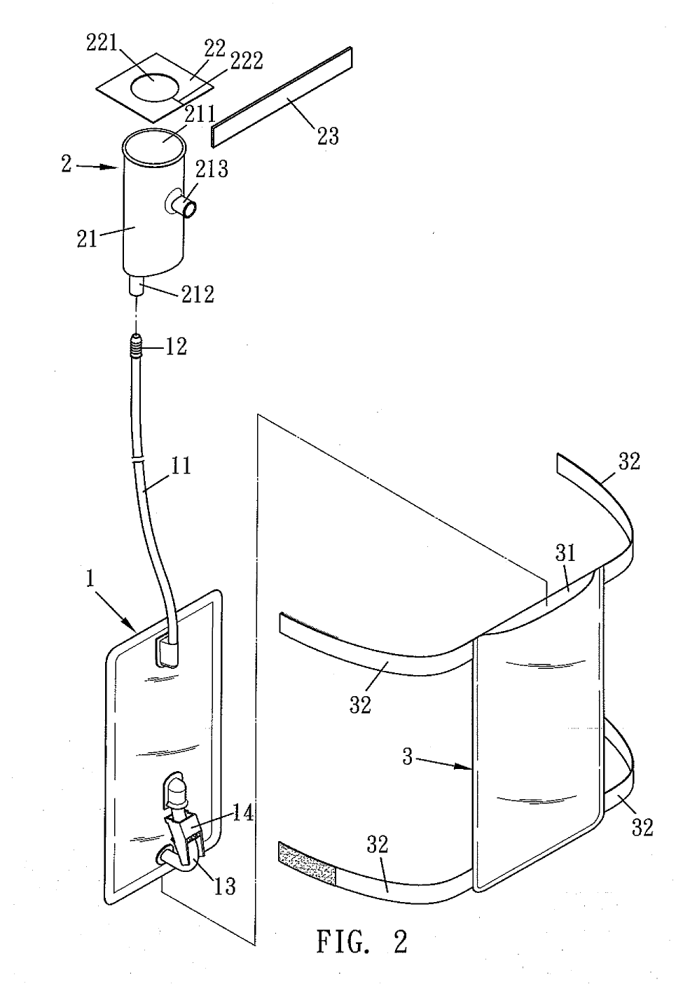

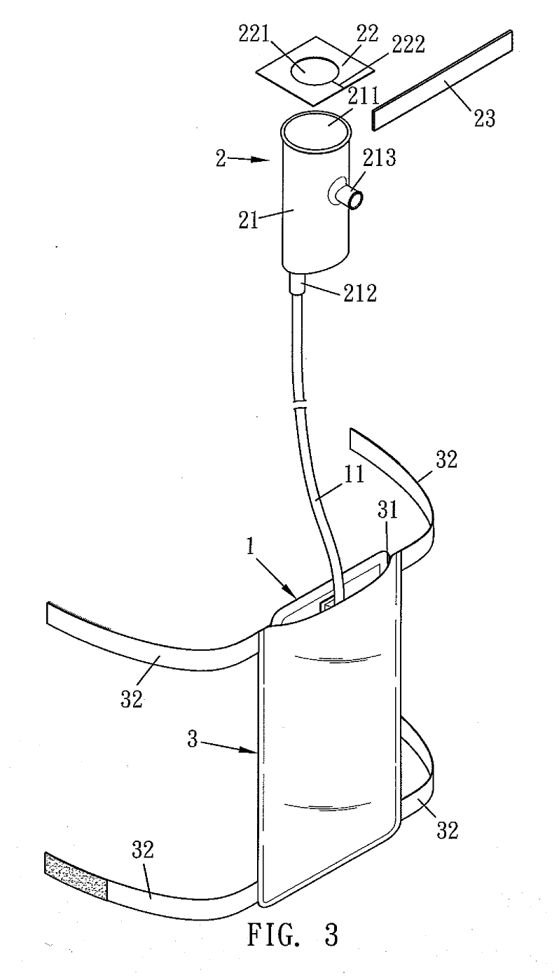

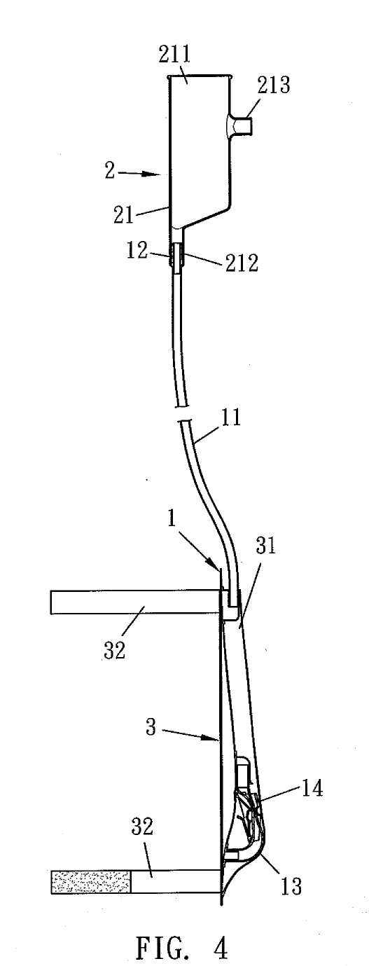

Referring to FIGS. 2-4, a catheterization device for a urine bag in accordance with the present invention comprises a urine bag 1 coupled with a sleeve unit 2 and received in a storage bag 3 for being attached onto a user's leg, wherein the urine bag 1 includes a conduit 11 connected on the top end thereof and having a connector 12 coupled onto one end thereof, and includes a tubular member 13 connected on the lower end thereof and having a control valve 14 mounted thereon for discharging the urine stored in the urine bag 1, and the sleeve unit 2 includes a sleeve body 21, a stop piece 22, and a double sided tape 23. The sleeve body 21 is made of a soft flexible material and formed in a cylinder shape, and includes an opening 211 arranged on one end thereof and a connecting post 212 extending from another end thereof, and includes a ventilation hose 213 extending from one side of the outer periphery thereof. The stop piece 22 is formed in the shape of a lamination and includes a through hole 221 arranged at the center thereof and a slot 222 disposed on one side thereof for communicating with the through hole 221, and the double sided tape 23 is made of anti-allergic material, such as emulsion, the storage bag 3 includes a bore 31 formed on the upper end thereof for receiving the urine bag 1, and includes two bands 32, each adhered on one side thereof.

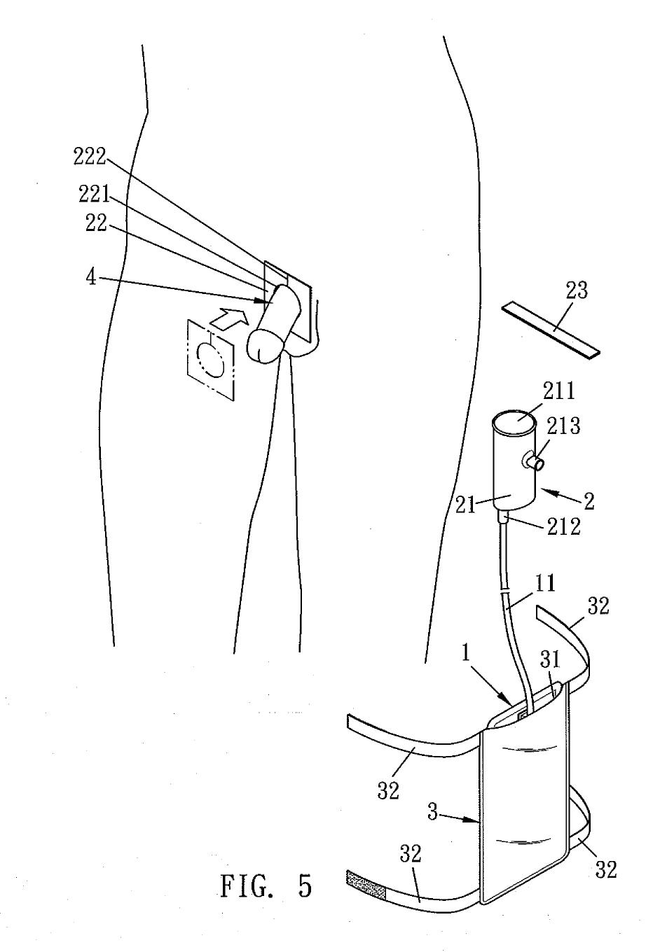

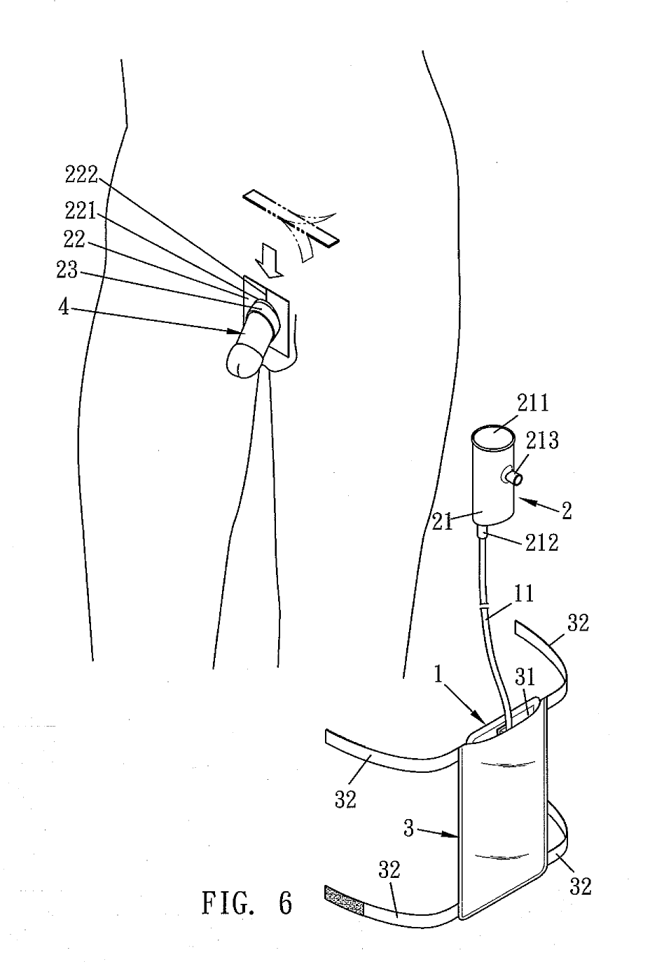

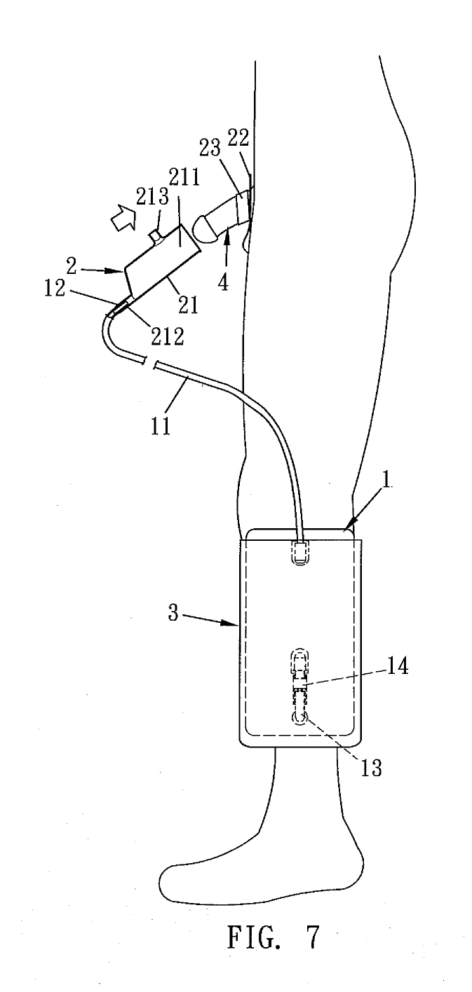

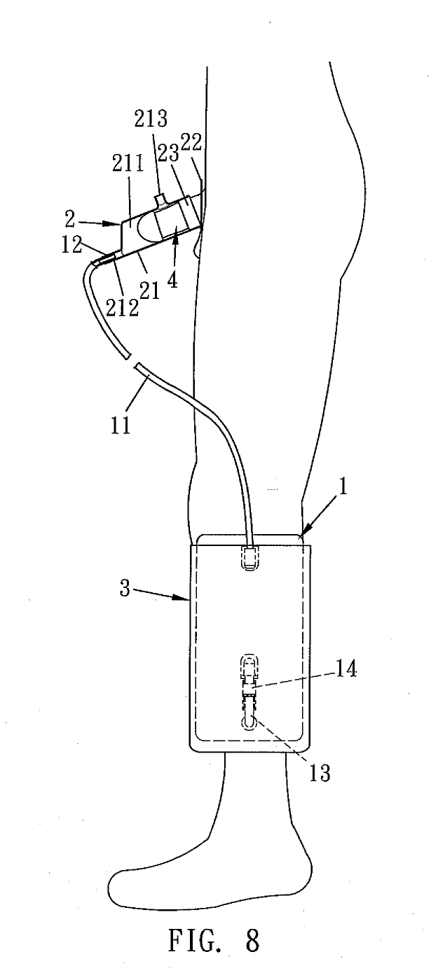

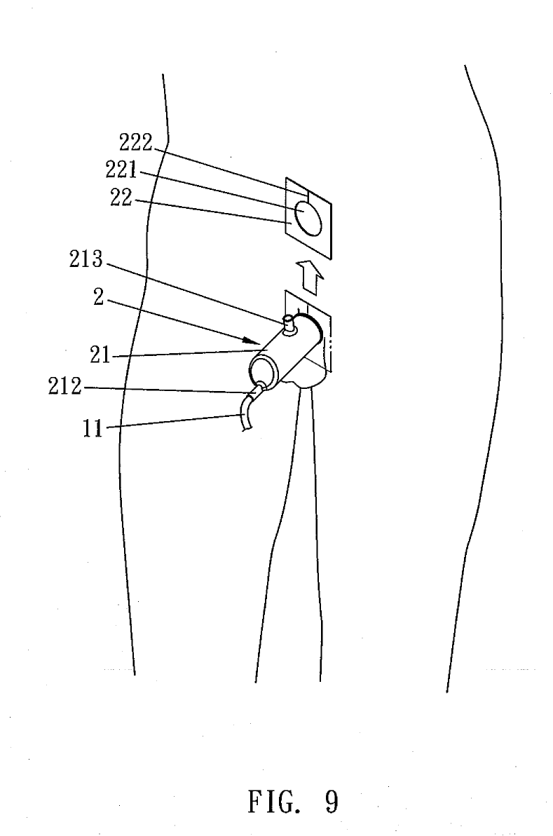

In operation, as illustrated in FIGS. 5-9, the stop piece 22 of the sleeve unit 2 is fitted onto the bottom of a male genital 4 by way of the through hole 221 so as to stop pubes, and the double sided tape 23 is surrounded and adhered on the male genital 4 ahead of the stop piece 22, such that the pubes are not adhered by the double sided tape 23. Thereafter, the sleeve body 21 is fitted onto the entire male genital 4 by using the opening 211 so that the opening 211 is adhered by the double sided tape 23, and then the stop piece 22 is removed from the bottom of the male genital 4.

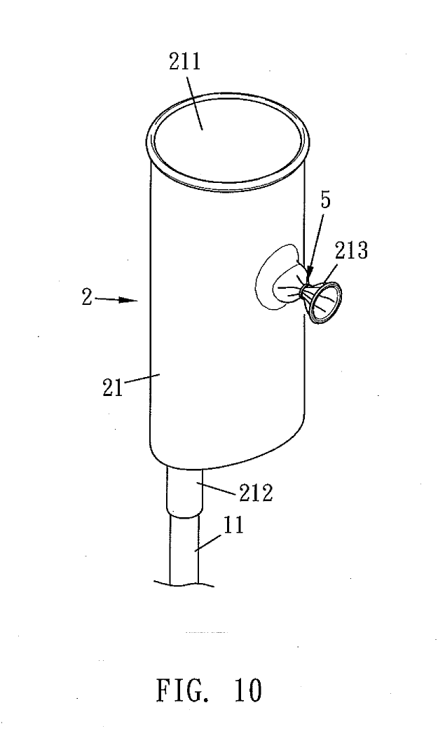

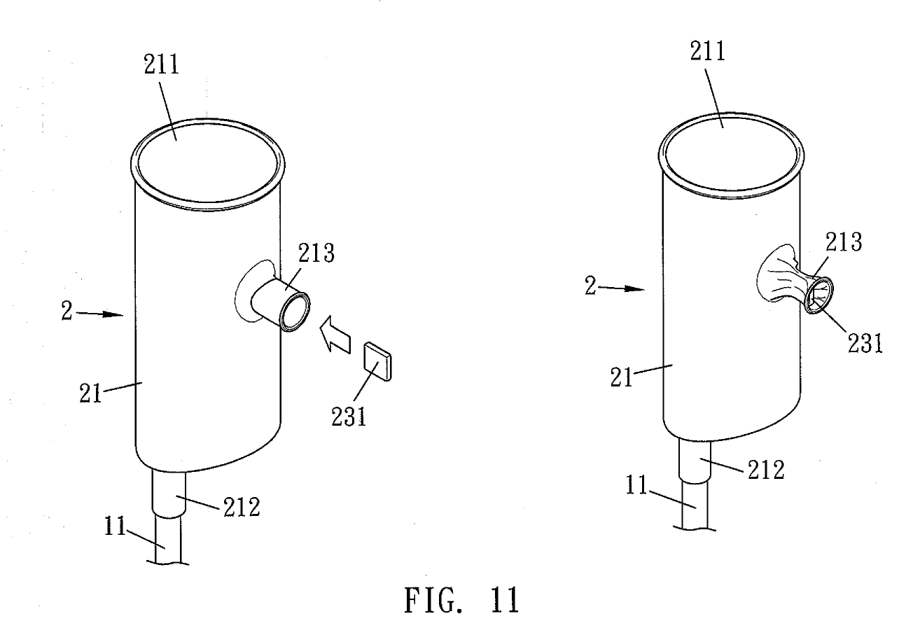

Referring further to FIGS. 10 and 11, in the closed state of the ventilation hose of the sleeve body, the sleeve body 21 of the sleeve unit 2 is fixed on the genital 4 securely, and the ventilation hose 213 allows to ventilate so that the urines flow into the urine bag 1 well in day time. On the other hand, as the user sleeps in night time, a tie member 5, a clamping member, or a torn segment 231 of the double sided tape 23 is provided to close the ventilation hose 213 completely so as to prevent from the leak of the urines.

It can be clearly seen from the preceding accounts on the features of the present invention that the catheterization device for the urine bag of the present invention has the following advantages:

1. The sleeve body 21 is made of soft flexible material, obtaining comfortness, and the double sided tape 23 may adhere the sleeve body 21 onto the male genital 4 securely. In addition, the ventilation hose 231 may prevent from a vacuum, discharging urines to the urine bag 1 effectively.

2. The air permeability is good because the sleeve member 4 includes a ventilation hose 213 extending from one side of the outer periphery thereof, thereby preventing from bacteria affection.

The invention is not limited to the above embodiment but various modifications thereof may be made. It will be understood by those skilled in the art that various changes in form and detail may be made without departing from the scope and spirit of the present invention.

What is claimed is:

1. A catheterization device for a urine bag comprising:
a urine bag coupled with a sleeve unit and including a conduit connected on the top end thereof and a tubular member connected on the lower end thereof and having a control valve mounted thereon, characterized in
said sleeve unit includes a sleeve body and a double sided tape, and said sleeve body is made of soft flexible material and includes an opening arranged on one end thereof and a connecting post extending from another end thereof for connecting with said conduit of said urine bag, and includes a ventilation hose extending from one side of the outer periphery thereof, said double sided tape is provided to adhere a male genital and said sleeve body.

2. The catheterization device for the urine bag as claimed in claim 1 further comprising a stop piece including a through hole arranged at the center thereof and a slot disposed on one side thereof for communicating with said through hole so as to stop pubes and be removed from the bottom of said male genital after fitting said sleeve body onto said entire male genital.

3. The catheterization device for the urine bag as claimed in claim 1, wherein said ventilation hose is closed completely so as to prevent from the leak of the urines.

4. The catheterization device for the urine bag as claimed in claim 3, wherein said ventilation hose is closed completely by a tie member.

5. The catheterization device for the urine bag as claimed in claim 3, wherein said ventilation hose is closed completely by a clamping member.

6. The catheterization device for the urine bag as claimed in claim 3, wherein said ventilation hose is closed completely by a torn segment of said double sided tape.
